(19) United States
(12) Patent Application Publication
(10) Pub. No.: US 2015/0100098 A1
(43) Pub. Date: Apr. 9, 2015

(54) ROD REDUCER

(71) Applicant: K2M, INC., Leesburg, VA (US)

(72) Inventor: Brandon Moore, Summit Point, WV (US)

(21) Appl. No.: 14/508,279

(22) Filed: Oct. 7, 2014

Related U.S. Application Data
(60) Provisional application No. 61/887,911, filed on Oct. 7, 2013.

(51) Int. Cl. A61B 17/70

(52) U.S. Cl. CPC A61B 17/7086; A61B 17/7002
USPC 606/86 A

(57) ABSTRACT

A rod reducer apparatus is disclosed and includes a housing, an anvil coupled to a shaft, a plurality of arm members, and a button. The housing has an opening extending therethrough for receiving the shaft. The anvil is coupled to one end of the shaft. The button is slidably disposed in the housing and transitionable between first and second positions. The button is engageable with the shaft such that rotation of the shaft is translated into linear movement of the anvil.

### CROSS-REFERENCE TO RELATED APPLICATIONS

The present application claims the benefit of U.S. Provisional Application Serial No. 61/887,911, which was filed on Oct. 7, 2013, the entire contents of which are incorporated herein by reference.

### BACKGROUND

1. Technical Field

The present disclosure relates to orthopedic surgery apparatus for stabilizing and fixing the bones and joints of the body. Particularly, the present disclosure relates to a manually operated apparatus for reducing a spinal rod into a bone screw in a controlled, measured, and efficient manner.

2. Description of Related Art

The spinal column is a complex system of bones and connective tissues that provides support for the human body and protection for the spinal cord and nerves. The human spine is comprised of thirty-three vertebrae at birth and twenty-four as a mature adult. Between each pair of vertebrae is an intervertebral disc, which maintains the space between adjacent vertebrae and acts as a cushion under compressive, bending, and rotational loads and motions.

There are various disorders, diseases, and types of injury that the spinal column may experience in a lifetime. The problems may include but are not limited to scoliosis, kyphosis, excessive lordosis, spondylolisthesis, slipped or ruptured disc, degenerative disc disease, vertebral body fracture, and tumors. Persons suffering from any of the above conditions typically experience extreme or debilitating pain and often times diminished nerve function.

One of the more common solutions to any of the above mentioned conditions involves a surgical procedure known as spinal fusion. A spinal fusion procedure involves fusing two or more vertebral bodies in order to stabilize or eliminate motion at the intervertebral disc or joint. To achieve this, natural or artificial bone, along with a spacing device, replaces either part, or the entire intervertebral disc to form a rigid column of bone, which is stabilized by mechanical hardware.

The mechanical hardware used to immobilize the spinal column typically involves a series of bone screws/anchors and metal rods or plates. When the spine surgery is performed posteriorly, it is common practice to place bone screws into the vertebral bodies and then connect a metal rod between adjacent vertebral bodies. When the spine surgery is performed anteriorly, it is common practice to attach a thin metal plate directly to the vertebral bodies and secure it to each vertebral level using one or more bone screws.

The process of properly inserting the spinal rod into the receiving slot of a bone screws and then securing that connecting rod in place can often require that the clinician use a number of instruments and expend a great deal of time and effort. When bone screws in several adjacent vertebrae are to be securely connected by a spinal rod, the repeated process of inserting the rod into the screw housing of the bone screws and then securing the rod in place for each respective bone screw can be difficult, tiresome, and time consuming. Further, the alignment of the rod as it connects to each of the sequential bone screws may require adjustment during the procedure and, therefore it is desirable that an apparatus and method be provided by which the rod can be reduced into the screw housing of each of the sequentially aligned bone screws and, as necessary, easily adjusted so as to facilitate the process for the clinician with minimal effort and loss of time. Therefore, a need exits for an efficient way to reduce the rod into the screw housing and lock the rod in place.

### SUMMARY

The present disclosure is directed to a rod reducer apparatus including a housing having an opening, a shaft disposed through the opening and having threads formed thereon, and an anvil coupled to the shaft. The rod reducer apparatus further includes a button slidably disposed in the housing which is transitionable between a first position, wherein the threads of the shaft are engaged with threads on an inner surface of the button, and a second position, wherein the threads of the shaft are spaced apart from the threads on the inner surface. Additionally, first and second arm members are coupled to the housing and are configured to engage a bone screw. First and second arms are movable towards a parallel configuration as the anvil is advanced away from the housing. The rod reducer apparatus is configured such that rotation of the shaft with the button in the first position translates into linear movement of the shaft relative to the housing, while linear movement of the shaft with the button in the second position is independent of shaft rotation.

In one embodiment, the rod reduction apparatus further includes an elongated throughhole that extends through the button, the elongated throughhole is alignable with the opening such that the shaft is insertable therethrough.

In one embodiment, the shaft cooperatively engages threads of the button with the button in the first position.

In a further embodiment, the diameter of the elongated throughhole is larger than a diameter of the shaft.

In yet another embodiment, the first and second arm members are pivotably coupled to the housing.

In a further embodiment, the first and second arm members are flexibly coupled to the housing.

In one embodiment, the rod reduction apparatus further includes a spring element disposed in the housing and abutting the button.

In yet another embodiment, the spring element biases the button towards the first position.

In a further embodiment, a receiving saddle is disposed on the anvil, such that the receiving saddle cooperatively engages with a surface of a spinal rod.

In another embodiment, the receiving saddle is generally formed into an arch, and is adapted to engage with a variety of spinal rod diameters.

In one embodiment, a head at a proximal end of the shaft is adapted to cooperatively engage with a drive tool.

In another aspect of the present disclosure, a method for reducing a spinal rod into a bone screw includes, providing a rod reducer apparatus including, a housing, a shaft disposed through the housing, and an anvil coupled to the shaft. The rod reducer apparatus further includes a button that is transitionable between a first position and a second position. Additionally, first and second arm members are coupled to the housing and are configured to engage the bone screw. The rod reducer apparatus is configured such that rotation of the shaft with the button in the first position translates into linear movement of the shaft relative to the housing, while linear movement of the shaft with the button in the second position is independent of shaft rotation. The method further includes coupling the rod reducer apparatus to the bone screw, positioning the spinal rod between the anvil, the first and second arm members, and the screw housing of the bone screw, transitioning the button of the rod reducer apparatus from the first position to the second position, sliding the shaft, and anvil attached thereto, distally such that the arm members grasp the bone screw, and sliding the shaft and anvil distally such that the anvil comes into contact with the spinal rod.

In one embodiment of the present disclosure, the method may further include, transitioning the button of the rod reducer apparatus to the first position and rotating the shaft such that the shaft and anvil travel linearly with respect to the housing towards the spinal rod such that the anvil urges the spinal rod into engagement with the screw housing of the bone screw.

In yet another embodiment of the present disclosure, the method may further include, manipulating the spinal rod and bone screw into a desired orientation with the anvil securely holding the spinal rod in engagement with the screw housing.

In yet another embodiment of the present disclosure, the method may further include, transitioning the button of the rod reducer apparatus to the second position and sliding the shaft away from the spinal rod.

In a further embodiment of the present disclosure, the method may further include, decoupling the first and second arm members of the rod reducer apparatus from the bone screw.

In an embodiment of the present disclosure, the method may further include, selecting the spinal rod from a plurality of spinal rods having varying diameters, selecting the bone screw from a plurality of bone screws having a variety of sizes, and reducing the selected spinal rod into the selected bone screw.

In a further embodiment of the present disclosure, the method may further include, implanting at least one bone screw into a bone of a subject.

In another aspect of the present disclosure, a kit is provided. The kit includes a rod reducer apparatus, a plurality of bone screws, and at least one spinal rod. The rod reducer apparatus includes, a housing having an opening, a shaft disposed through the opening, and an anvil coupled to the shaft. The rod reducer apparatus further includes a button slidably disposed in the housing which is transitionable between a first position and a second position. Additionally, first and second arm members are coupled to the housing and are configured to engage a bone screw. The rod reducer apparatus is configured such that rotation of the shaft with the button in the first position translates into linear movement of the shaft relative to the housing, while linear movement of the shaft with the button in the second position is independent of shaft rotation.

### BRIEF DESCRIPTION OF THE DRAWINGS

The above and other aspects and features of the present disclosure will become more apparent in light of the following detailed description when taken in conjunction with the accompanying drawings in which:

FIG. 1 is a front view of one embodiment of a rod reducer apparatus, in a first orientation, in accordance with the present disclosure;

FIG. 2 is a front view of the rod reducer apparatus of FIG. 1 in a second orientation;

FIG. 3 is an perspective view, with parts separated, of the rod reducer apparatus of FIGS. 1 and 2;

FIG. 4A is a front perspective view of one embodiment of a housing of the rod reducer apparatus of FIG. 1 in accordance with the present disclosure;

FIG. 4B is a rear perspective view of the housing of FIG. 4A;

FIG. 5 a perspective view of one embodiment of a button of the rod reducer apparatus of FIG. 1 in accordance with the present disclosure;

FIG. 6 is a perspective view of an arm member of the rod reducer apparatus of FIG. 1 in accordance with the present disclosure;

FIG. 7 is a perspective view of an anvil of the rod reducer apparatus of FIG. 1 in accordance with the present disclosure;

FIG. 8 is a cross-sectional view of the rod reducer apparatus of FIG. 2 in accordance with the present disclosure;

FIG. 9A is a perspective view of the rod reducer apparatus of FIG. 1 coupled to a bone screw prior to reducing a rod; and

FIG. 9B is a perspective view of the rod reducer apparatus of FIG. 9A after reducing the rod into the bone screw.

### DETAILED DESCRIPTION OF EMBODIMENTS

Embodiments of the present disclosure are now described in detail with reference to the drawings in which like reference numerals designate identical or corresponding elements in each of the several views. As used herein, the term “clinician” refers to a doctor, a nurse, or any other care provider and may include support personnel. Throughout this description, the term “proximal” will refer to the portion of the apparatus or component thereof that is closer to the clinician and the term “distal” will refer to the portion of the apparatus or component thereof that is farther from the clinician. In addition, the term “cephalad” is used in this application to indicate a direction toward a patient's head, whereas the term “caudad” indicates a direction toward the patient's feet. Further still, for the purposes of this application, the term “lateral” indicates a direction toward a side of the body of the patient, i.e., away from the middle of the body of the patient, whereas “medial” refers to a position toward the middle of the body of the patient. The term “posterior” indicates a direction toward the patient's back, and the term “anterior” indicates a direction toward the patient's front. Additionally, in the drawings and in the description that follows, terms such as front, rear, upper, lower, top, bottom, and similar directional terms are used simply for convenience of description and are not intended to limit the disclosure.

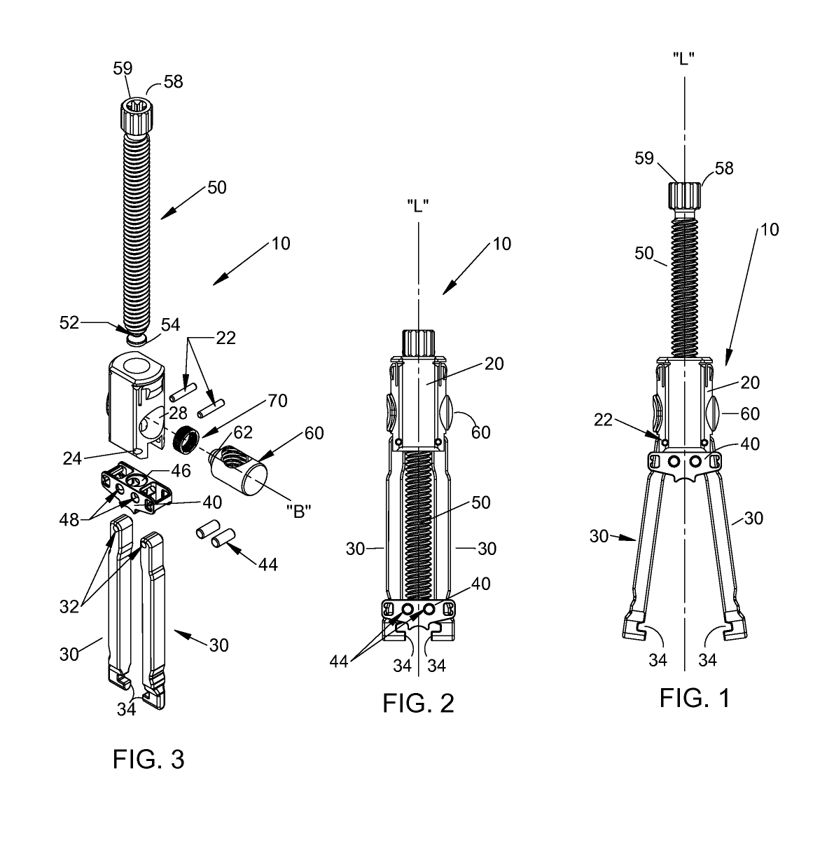

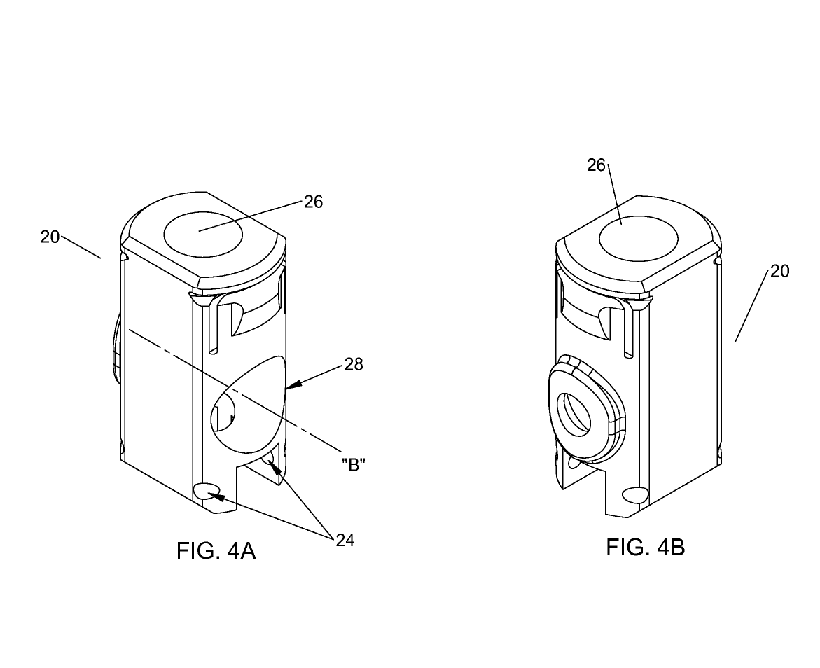

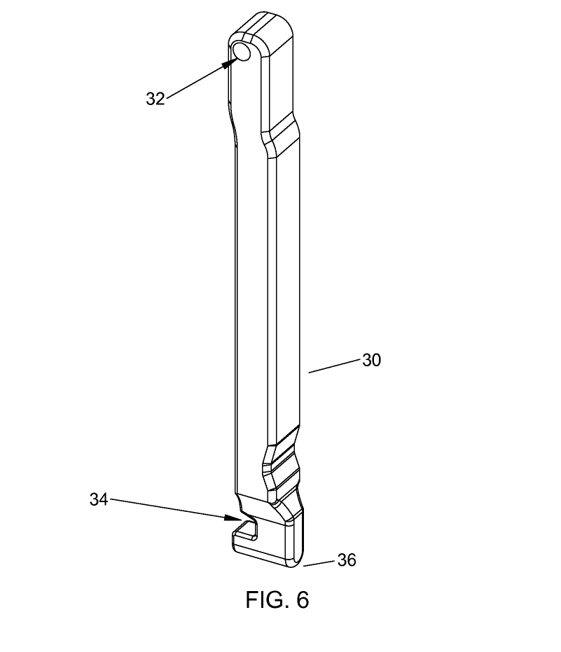

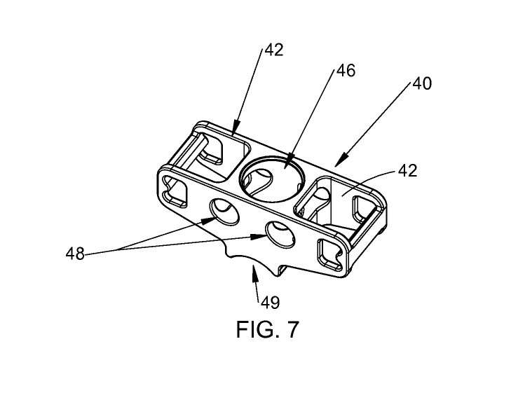

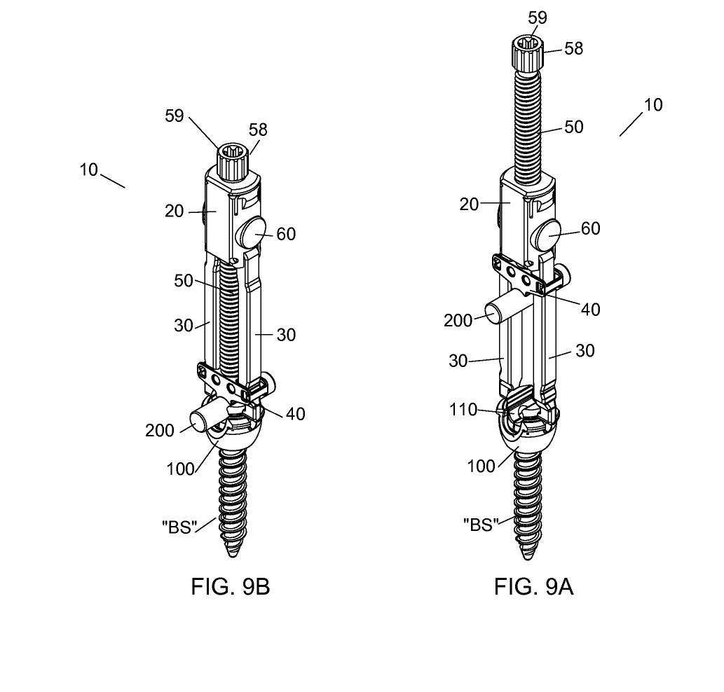

Referring initially to FIGS. 1-3, a rod reducer in accordance with the present disclosure is generally designated as 10. Rod reducer 10 includes a housing 20, a plurality of arm members 30, an anvil 40 coupled to a shaft 50, and a button 60. With further reference to FIGS. 4A, 4B and 7, rod reducer 10 may include two arm members 30. Each arm member 30 is insertable through a respective cavity 42 of the anvil 40. Arm members 30 are pinned in place relative to housing 20 with pins 22. Alternatively, it is contemplated that arm members 30 may be integrally formed with housing 20 such that, rather than pivoting relative to housing 20, arm members 30 flex relative to housing 20. In such an embodiment, pins 22 may be omitted and arms 30 may be directly attached to housing 20. Pins 22 extend through a respective pin hole 24 of the housing 20 and a respective pin hole 32 of each arm member 30. Pin holes 32 in combination with pins 22 and pin holes 24 define a pivot axis for first and second arm members 30. As seen in FIG. 6, each arm member 30 has a hook portion 34 at its distal end 36 for engaging a screw housing 100 that is disposed at a proximal end of a bone screw “BS” (as seen in FIGS. 9A and 9B). During reduction of a spinal rod 200, arm members 30 move towards a parallel configuration, such that hook portion 34 of each respective arm member 30 may engage bone screw “BS” (as seen in FIGS. 9A and 9B). Engagement of hook portion 34 to bone screw “BS” serves to maintain alignment of rod reducer 10 with respect to the screw housing 100 as spinal rod 200 is reduced into the screw housing 100.

With reference to FIGS. 1 and 2, anvil 40 and arm members 30 will be further described. Proximal and distal translation of anvil 40 causes each arm member 30 to pivot with respect to housing 20 about their respective pin holes 32. In an alternate embodiment, each arm member 30 flexes relative to housing 20 as anvil 40 is translated proximally and distally with respect to housing 20. As seen in FIG. 1, with anvil 40 in a proximal most position, arm members 30 are in a first position, and may be engaged or unengaged from screw housing 100 of bone screw “BS”. As seen in FIG. 2, with anvil 40 in a distal most position, arm members 30 are in a second position, and are configured to be securely engaged with the screw housing 100 of bone screw “BS” (FIG. 9B). As anvil 40 travels distally with respect to housing 20 from the proximal most position to the distal most position, arm members 30 move from the first position towards a parallel configuration ending in the second position. Once arm members 30 move towards a parallel configuration, hook portion 34 of each respective arm member 30 acts to engage the screw housing 100 of bone screw “BS” (as discussed above and seen in FIGS. 9A and 9B).

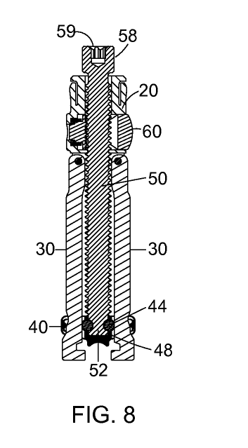

With reference to FIGS. 3, 7, and 8, the coupling of anvil 40 to shaft 50 of rod reducer 10 will be described. Shaft 50 has threads thereon and includes a distal portion 54 with an annular groove 52 and a proximal portion 58 with a head 59. It is envisioned that head 59 may be configured to cooperatively engage with any number of counterpart drive tools known in the art to effect torque driven rotation. For example, head 59 may be configured to receive a hex head (as shown in FIG. 3) or a Philips or slotted screwdriver. Shaft 50 is insertable through aperture 46 of anvil 40. Pins 44 are used to maintain the shaft 50 within the anvil 40 by inserting pins 44 through pin holes 48 of anvil 40 such that a portion of each pin 44 resides in the annular groove 52 at the distal end 54 of shaft 50.

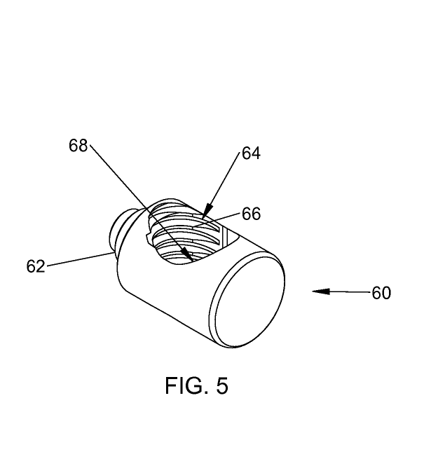

With reference to FIGS. 1-5, the housing 20 and button 60 will be further described. Housing 20 of rod reducer 10 includes an opening 26. In one embodiment of the present disclosure, housing 20 defines a longitudinal axis “L” (as seen in FIGS. 1 and 2) such that opening 26 coincides with longitudinal axis “L”. Housing 20 further includes a button hole 28 disposed thereon configured to receive button 60. Button 60 may have an elongated throughhole 64 extending therethrough (as seen in FIG. 5) which is alignable with the opening 26 of housing 20 such that shaft 50 is insertable therethrough. It is envisioned that button 60 may be held in place within button hole 28 by the shaft 50 passing through the opening 26 of housing 20 and the elongated throughhole 64 of button 60. It is further envisioned that elongated throughhole 64 has a diameter which is larger than a diameter of shaft 50.

With further reference to FIGS. 3 and 5, engagement and disengagement of button 60 to shaft 50 will be described. In one embodiment of the present disclosure, an inner surface of the elongated throughhole 64 has a partially threaded portion 66 disposed thereon configured to receive shaft 50. The inner surface of the elongated throughhole 64 also has an unthreaded portion 68 to permit shaft 50 to slide freely therethrough. It is further envisioned that the elongated throughhole 64 may not be perfectly round, but may have an elongated or oval configuration, such that movement of the button 60 along axis “B” (as shown in FIGS. 3 and 4A) within the button hole 28 of housing 20 moves the threaded portion 66 of button 60 into and out of engagement with shaft 50. With button 60 in a first position, threaded portion 66 of button 60 is coupled to the shaft 50, permitting torque driven rotation and proximal and distal translation of the shaft 50 within the opening 26 (as seen in FIGS. 4A and 4B) of the housing 20. In a second position, threaded portion 66 of button 60 is uncoupled from shaft 50 permitting free movement of the shaft 50 within the opening 26 of the housing 20. It is envisioned that unthreaded potion 68 of the button 60 may be in near abutment to shaft 50 in the second position. In other words, with threaded portion 66 engaged to shaft 50, proximal and distal movement of shaft 50 and anvil 40 is directly proportional to the threaded configuration of the shaft 50 and the threaded portion 66, and may be thought of as a fine adjustment during reduction of a spinal rod. Further, with threaded portion 66 disengaged from shaft 50, proximal and distal movement of shaft 50 is not constrained allowing rapid movement of the shaft 50 and anvil 40 relative to the housing 20, and may be thought of as a course adjustment during reduction of a spinal rod.

With reference to FIGS. 9A and 9B, the movement of the shaft 50 and anvil 40 will be further discussed with respect to the engagement and disengagement of the button 60. During engagement of button 60 and torque driven rotation of shaft 50, anvil 40 may travel towards and away from housing 20 in unison with the proximal and distal translation of shaft 50. During reduction of spinal rod 200 into bone screw “BS”, threaded rod 50 is manually rotated distally such that anvil 40 simultaneously travels distally with respect to housing 20 into contact with spinal rod 200 to drive spinal rod 200 securely into screw housing 100. With button 60 in the second position, threaded rod 50 can slide freely both proximally and distally within opening 26 of housing 20 causing anvil 40 to simultaneously move freely with respect to housing 20 in the proximal and distal directions.

As illustrated in FIG. 3, a spring 70 may be disposed between button 60 and a spring seat (not shown) in housing 20. It is further envisioned that spring 70 may fit around a stub 62 of the button 60, or may alternatively seat in a recess (not shown) of the housing 20. Spring 70 and button 60 are inserted into button hole 28 of housing 20, and may be held in place by the shaft 50 passing through the opening 26 of housing 20 and the elongated throughhole 64 of button 60. Spring 70 provides a biasing force that urges button 60 towards the first position, keeping the threaded portion 66 of button 60 coupled to the shaft 50. In a contemplated alternative, spring 70 provides a biasing force which urges button 60 into the second position, keeping the threaded portion 66 of button 60 uncoupled from the shaft 50. In either embodiment, the biasing force of spring 70 may be overcome to achieve the desired button position, allowing either manual rotation of shaft 50 within opening 26 of housing 20 in the first position, or conversely, allowing shaft 50 to freely slide within the opening 26 of housing 20 in the second position.

With reference to FIG. 7, anvil 40 and spinal rod 200 will be further described. During reduction of spinal rod 200, receiving saddle 49 of anvil 40 is in abutment to an outer surface (not shown) of spinal rod 200. It is envisioned that receiving saddle 49 is configured to accommodate a range of spinal rod diameters. For example, receiving saddle 49 may be adapted to cooperatively engage with a spinal rod 200 having a variance in diameter of approximate 3 mm to 8 mm, while still achieving the necessary driving force to secure the spinal rod 200 into a bone screw “BS”. Receiving saddle 49 may be generally arched or convex, but may take the form of any geometric shape adapted to cooperatively engage with and drive a spinal rod during reduction.

Operating a rod reduction apparatus in accordance with the present disclosure will be described with reference to FIGS. 1-9B. A spinal rod and screw construct is assembled in a patient as follows. A clinician implants a bone screw “BS” into a spinal vertebra with a screw housings 100 of the bone screw “BS” positioned to receive a spinal rod 200 in a rod retaining seat or saddle portion 110 of the screw housing 100. It is envisioned that a clinician may implant multiple bone screw “BS” into several vertebra during a procedure. Once the desired number of bone screws “BS” have been implanted, the clinician aligns and manipulates the spinal rod 200 such that a portion of the spinal rod 200 is in proximal relation to the screw housing 100 of each respective bone screws “BS”, such that spinal rod 200 creates an unbroken connection between each bone screw “BS”.

The clinician next positions a rod reducer apparatus 10 into proximity with each respective bone screw “BS”, such that a hook portion 34 of arm members 30 of rod reducer 10 is in near abutment to the screw housing 100 of each respective bone screw “BS”. Next, the clinician causes the hook portion 34 of the arm members 30 to grasp, clip, or otherwise affix to the screw housing 100, such that during reduction of spinal rod 200 attachment of the rod reducer 10 to the bone screw “BS”, and alignment of spinal rod 200 to the screw housing 100, is maintained. During reduction, spinal rod 200 is positioned between the screw housing 100, the anvil 40, and the arm members 30, and may be in abutment to the anvil 40 (as seen in FIG. 9A) or in abutment to the screw housing 100 (as seen in FIG. 9B).

The clinician next reduces spinal rod 200 into seat 110 of screw housing 100. Often times there may be 15 mm or more of travel required in order to reduce spinal rod 200 fully within the seat 110 of screw housing 100 such that spinal rod 200 and screw housing 100 can be locked. Manually rotating threaded rod 50 such a distance can be cumbersome, tedious, and time consuming. The second position of button 60 of rod reducer 10 permits the clinician to perform course adjustments and quickly slide shaft 50 distally to position anvil 40 against spinal rod 200 to effect a reduction of spinal rod 200 into screw housing 100. With button 60 in the second position, shaft 50 is uncoupled from a threaded portion 66 of an elongate hole 64 disposed on the button 60, allowing shaft 50 to freely slid distally through the opening 26 of the housing 20. Free translation of shaft 50 permits anvil 40 to rapidly move distally in relation to housing 20 towards and into abutment with spinal rod 200. In the envisioned method of reducing a spinal rod, with button 60 in the second position, the clinician freely slides shaft 50 through the opening 26 of housing 20 until anvil 40 abuts spinal rod 200. The clinician continues to slide threads shaft 50, and anvil 40 attached thereto, distally causing spinal rod 200 into near abutment with screw housing 100. With a plurality of rod reducer apparatus 10, where each rod reducer apparatus 10 is mounted to a different bone screw “BS”, the clinician is able to gradually reduce the spinal rod 200 to a plurality of bone screws “BS” by sequentially reducing each rod reducer apparatus 10 all or part way until all rod reducer apparatus 10 have been actuated fully and the spinal rod 200 is reduced into all of the adjacent bone screws “BS”.

Once the anvil 40 is in abutment to spinal rod 200, and/or spinal rod 200 is in abutment to screw housing 100, the clinician may move button 60 into the first position. In the first position, shaft 50 is coupled to the threaded portion 66 of the elongated throughhole 64 of the button 60, such that the clinician may make fine adjustments and manually, or with a surgical tool (not shown), rotate shaft 50 causing anvil 40 to drive spinal rod 200 into securement with screw housing 100. In the first position, the clinician is provided a mechanical advantage of torque driven rotation of spinal rod 50.

With the rod reducer 10 attached to bone screw “BS”, it is further envisioned that the clinician may additionally use rod reducer 10 to further assist the alignment of spinal rod 200 between multiple bone screws “BS”. The clinician is provided a mechanical advantage to further bend or shape spinal rod 200 while spinal rod 200 is securely held by both rod reducer 10 and the screw housing 100 of the bone screw “BS”. In this configuration, the clinician may make final adjustments to the spinal rod 200 when connecting spinal rod 200 between multiple bone screws “BS”. After spinal rod 200 is properly aligned, the clinician may further reduce spinal rod 200 to secure the spinal rod 200 into the screw housing 100 of the bone screw “BS”.

Upon final alignment of spinal rod 200 between multiple bone screws “BS”, and/or securement of spinal rod 200 into screw housing 100, the clinician may place button 60 into the second position. In the second position, shaft 50 can again slide freely within the opening 26 of the housing 20, to permit anvil 40 to quickly and easily move proximally with respect to housing 20. Once the clinician moves shaft 50 and anvil 40 into a proximal most position (as seen in FIG. 1), arm members 30 of rod reducer 10 may be decoupled from the screw housing 100, permitting the clinician to detach rod reducer 10 from the bone screw “BS”.

In accordance with the present disclosure, it is envisioned that the clinician may perform the method described above with multiple bone screws “BS”, implanted in sequence to a number of vertebra, to facilitate the reduction of spinal rod 200 into and between multiple screw housings 100. It is envisioned that the clinician may be provided with multiple spinal rods 200. The clinician may perform the method described above to facilitate the reduction of multiple spinal rods 200 into multiple screw housings 100 to a number of vertebras in sequence. It is further envisioned that the clinician may be provided with multiple bone screws and spinal rods of varying sizes.

In accordance with the present disclosure, a kit will be described with reference to FIGS. 1-9B. The kit includes a rod reducer 10 in a package (not shown). The kit may further include a bone screw “BS”, a spinal rod 200, an orthopedic tool or device (not shown), and instructions for use. Examples of the orthopedic tool or device may be a tightening or loosening tool, an alignment tube, or a locking device. It is further envisioned, that the kit may include multiple rod reducer apparatus 10, multiple bone screws “BS”, and multiple spinal rods 200. Further, the kit may include a variety of sizes of bone screws “BS” and spinal rods 200. The package may include a thermoformed plastic tray and/or other packaging materials within the view of those skilled in the art.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of presently disclosed embodiments. Thus, the scope of the embodiments should be determined by the claims of the present application and their legal equivalents, rather than by the examples given.

What is claimed is:

1. A rod reducer apparatus comprising:
a housing having an opening;
a shaft disposed through the opening, the shaft having threads formed thereon;
an anvil coupled to the shaft;
a button slidably disposed in the housing and transitionable between a first position wherein the threads of the shaft are engaged with threads on an inner surface of the button and a second position wherein the threads of the shaft are spaced apart from the threads on the inner surface; and
first and second arm members coupled to the housing, the first and second arm members configured to engage a bone screw, the first and second arms movable towards a parallel configuration as the anvil is advanced away from the housing,
wherein rotation of the shaft with the button in the first position translates into linear movement of the shaft relative to the housing and linear movement of the shaft with the button in the second position is independent of shaft rotation.

2. The rod reducer apparatus of claim 1, further comprising an elongated throughhole extending through the button, the elongated throughhole alignable with the opening such that the shaft is insertable therethrough.

3. The rod reducer apparatus of claim 1, wherein the shaft cooperatively engages threads of the button with the button in the first position.

4. The rod reducer apparatus of claim 2, wherein a diameter of the elongated throughhole is larger than a diameter of the shaft.

5. The rod reducer apparatus of claim 1, wherein the first and second arm members are pivotably coupled to the housing.

6. The rod reducer apparatus of claim 1, wherein the first and second arm members are flexibly coupled to the housing.

7. The rod reducer apparatus of claim 1, further comprising a spring element disposed in the housing and abutting the button.

8. The rod reducer apparatus of claim 7, wherein the spring element biases the button towards the first position.

9. The rod reducer apparatus of claim 1, further comprising a receiving saddle disposed on the anvil, such that the receiving saddle cooperatively engages with a surface of a spinal rod.

10. The rod reduction apparatus of claim 9, wherein the receiving saddle is generally formed into an arch, and is adapted to engage with a variety of spinal rod diameters.

11. The rod reduction apparatus of claim 1, wherein a head at a proximal end of the shaft is adapted to cooperatively engage with a drive tool.

12. A method of reducing a spinal rod into a bone screw comprising:
providing a rod reducer apparatus including:
a housing;
a shaft disposed through the housing;
an anvil coupled to the shaft;
a button transitionable between a first position and a second position; and
first and second arm members coupled to the housing and configured to engage a bone screw, wherein rotation of the shaft with the button in the first position translates into linear movement of the shaft relative to the housing and linear movement of the shaft with the button in the second position is independent of shaft rotation;
coupling the rod reducer apparatus to the bone screw;
positioning the spinal rod between the anvil, the first and second arm members, and the screw housing of the bone screw;
transitioning the button of the rod reducer apparatus from the first position to the second position;
sliding the shaft, and anvil attached thereto, distally such that the arm members grasp the bone screw; and
sliding the shaft and anvil distally such that the anvil comes into contact with the spinal rod.

13. The method of claim 12, further comprising:
transitioning the button of the rod reducer apparatus to the first position; and
rotating the shaft such that the shaft and anvil travel linearly with respect to the housing towards the spinal rod such that the anvil urges the spinal rod into engagement with the screw housing of the bone screw.

14. The method of claim 13, further comprising:
manipulating the spinal rod and bone screw into a desired orientation with the anvil securely holding the spinal rod in engagement with the screw housing.

15. The method of claim 14, further comprising:
transitioning the button of the rod reducer apparatus to the second position; and
sliding the shaft away from the spinal rod.

16. The method of claim 15, further comprising:
decoupling the first and second arm members of the rod reducer apparatus from the bone screw.

17. The method of claim 12, further comprising:
selecting the spinal rod from a plurality of spinal rods having varying diameters;
selecting the bone screw from a plurality of bone screws having a variety of sizes; and
reducing the selected spinal rod into the selected bone screw.

18. The method of claim 12, further comprising implanting at least one bone screw into a bone of a subject.

19. A kit comprising:
a rod reducer apparatus including,
a housing having an opening;
a shaft disposed through the opening;
an anvil coupled to the shaft;
a button slidably disposed in the housing and transitionable between a first position and a second position; and
first and second arm members coupled to the housing and configured to engage a bone screw, wherein rotation of the shaft with the button in the first position translates into linear movement of the shaft relative to the housing and linear movement of the shaft with the button in the second position is independent of shaft rotation;
a plurality of bone screws; and
at least one spinal rod.
